(19) United States
(12) Patent Application Publication
(10) Pub. No.: US 2023/0207086 A1
(43) Pub. Date: Jun. 29, 2023

(54) METHOD AND SYSTEM BASED ON MATRIX CHECK FOR RAPIDLY DETERMINING RATIONAL USE OF MEDICINE

(71) Applicants: HOSPITAL OF CHENGDU OFFICE OF PEOPLE?S GOVERNMENT OF TIBETAN AUTONOMOUS REGION (HOSPITAL.C.T.), Chengdu (CN); MEDISOFTWARE CO., LTD., Chengdu (CN)

(72) Inventors: Xiaosi LI, Chengdu (CN); Yihong XIE, Chengdu (CN); Yong CHEN, Chengdu (CN); Yuru HE, Chengdu (CN); Shu ZHANG, Chengdu (CN); Guangrong TANG, Chengdu (CN); Rongcier LU, Chengdu (CN)

(73) Assignees: HOSPITAL OF CHENGDU OFFICE OF PEOPLE?S GOVERNMENT OF TIBETAN AUTONOMOUS REGION (HOSPITAL.C.T.), Chengdu (CN); MEDISOFTWARE CO., LTD., Chengdu (CN)

(21) Appl. No.: 17/971,669

(22) Filed: Oct. 24, 2022

(30) Foreign Application Priority Data
Dec. 27, 2021 (CN) 202111606884.1

(51) Int. Cl. G16H 20/10; G06F 16/903; G16H 70/40

(52) U.S. Cl. CPC G16H 20/10; G06F 16/90335; G16H 70/40; G06F 16/9027

(57) ABSTRACT

A method and a system based on matrix check for rapidly determining rational use of a medicine are provided. Attribute values of a medicine are established. The medicine attribute values are added to nodes starting from a root node. A range of attribute values is set, to obtain a determination standard for the attribute values. Data in a tree structure is obtained, and then is split according to different dimensions. A value is defined for each of the different dimensions. The tree structure is traversed to obtain values of each two attributes in the different dimensions. The obtained values of different dimensions are marked as subscripts of a multi-dimensional array. A check matrix is constructed by using the multi-dimensional array. A value of an element in the check matrix is extracted. A conclusion about rational use of the medicine is outputted based on the value of the obtained element.

### CROSS REFERENCE OF RELATED APPLICATION

The present application claims priority to Chinese Patent Application No. 202111606884.1, titled “METHOD AND SYSTEM BASED ON MATRIX CHECK FOR RAPIDLY DETERMINING RATIONAL USE OF MEDICINE”, filed on Dec. 27, 2021 with the China National Intellectual Property Administration, which is incorporated herein by reference in its entirety.

### FIELD

The present disclosure relates to the technical field of medical analysis, and in particular to a method and a system based on matrix check for rapidly determining rational use of a medicine.

### BACKGROUND

With the accelerated pace of urban modernization, medical safety has attracted much attention. It is particularly important to strengthen management of rational use of medicines and ensure standardized use of medicines.

In order to further improve the level of rational use of medicines in clinical practice, improve the therapeutic effect of diseases, and reduce health hazards and excessive economic burdens of patients due to irrational use of medicines, a system for rational use of medicines is constructed by means of information technology, so that patients can obtain the best therapeutic effect with the minimum treatment risk.

At present, the method for determining rational use of medicines is to simply establish a relationship between a medicine and the standard name as a basis for determining whether a diagnosis for use of medicines is in line with a symptom of a patient. This is difficult to apply to a case in which contraindications and applicable conditions of use and dosage of medicines are varying in the case of combined use of multiple medicines with various instructions.

In view of this, a method based on matrix check for rapidly determining rational use of medicines is proposed, helping a doctor to establish monitoring rules of rational use of a medicine efficiently and accurately. Further, a prescription ordered by a doctor is monitored, so as to achieve safe, reasonable and effective use of various medicines to patients.

### SUMMARY

In order to solve the above technical problems, a method based on matrix check for rapidly determining rational use of a medicine is provided according to technical solutions of the present disclosure. The method includes the following steps S11 to S16.

In step S11, attribute values of the medicine are established.

In step S12, a rule construction framework in a tree structure is established to obtain data in a tree structure as follows. The attribute values of the medicine are added to nodes starting from a root node. A range of the attribute values is set, to obtain a determination standard for the attribute values of the medicine. The data in a tree structure is obtained.

In step S13, the data in a tree structure is split according to different dimensions.

In step S14, a value is defined for each of the different dimensions.

In step S15, the tree structure is traversed to obtain values of each two attributes in the different dimensions.

In step S16, the obtained values of different dimensions are marked as subscripts of a multi-dimensional array. A check matrix is constructed by using the multi-dimensional array. A value of an element in the check matrix is extracted. A determination conclusion about rational use of the medicine is outputted based on the value of the obtained element.

In an embodiment, the attribute values of the medicine include rules of use of the medicine and conditions of use of the medicine.

In an embodiment, the determination standard for the attribute values of the medicine includes: a text class, a numerical interval class and an object class.

In an embodiment, the data in a tree structure is split according to the following dimensions 1 to 4.

The dimension 1 represents a node relationship, including: a peer node relationship, a sibling node relationship, a parent-child node relationship, and a condition-rule node relationship.

The dimension 2 represents a type of each of the attribute values, including: a character type, a numerical type, an object type, and a combination type. The character type is for representing an attribute value with a string set. The numerical type is for representing an attribute value with an interval. The object type is for representing an attribute value with multiple groups of key-value. The combination type is for representing an attribute value with multiple groups of intervals or sets.

The dimension 3 represents an operator relationship, including: element of & not element of, element of & element of, not element of & not element of, and element of & equality.

The dimension 4 represents an interval or set relationship, including: non-intersection, intersection, subset, equality.

In an embodiment, the value is defined for each of the different dimensions as follows.

For the dimension 1, a value is directly assigned to each relationship, to obtain an array R1.

For the dimension 2, coding is performed for different types of attribute values to obtain an array R2.

For the dimension 3, values are assigned to operators in censorship rules. Numerical tokens of two operators are added by concatenating strings. The combined numbers are sorted in ascending order to obtain an ordered array. A subscript of the ordered array represents a dimension value of an operation relation, to obtain an array R3.

For the dimension 4, a value is assigned to a relationship between two values of a same attribute to obtain R4.

In an embodiment, S16 includes: calculating values in the 4 dimensions; acquiring element values of a 4-dimensional array; inferring a verification result based on the element values of the array, and outputting the determination conclusion.

In an embodiment, a system based on matrix check for rapidly determining rational use of a medicine includes a data establishing module, a data analysis module, a check matrix constructing module and a prompt module for rational use of medicines. The data constructing module is configured to establish a rule construction framework in a tree structure to obtain data in a tree structure. The data analysis module is configured to split the data in a tree structure according to different dimensions, define a value for each of the different dimensions, and traverse the tree structure to obtain values of each two attributes in the different dimensions. The check matrix constructing module is configured to mark the obtained values of different dimensions as subscripts of a multi-dimensional array, and construct a check matrix by using the multi-dimensional array. The prompt module for rational use of medicines is configured to extract a value of an element in the check matrix, and output a determination conclusion about rational use of the medicine based on the value of the obtained element.

Compared with the existing technology, the present disclosure has the following beneficial effects. In the present disclosure, attribute values of a medicine are established, so that a rule construction framework in a tree structure is established to obtain data in a tree structure as follows. The medicine attribute values are added to nodes starting from a root node. A range of attribute values is set, to obtain a determination standard for the attribute values. The data in a tree structure is obtained. The data in a tree structure is split according to different dimensions. A value is defined for each of the different dimensions. The tree structure is traversed to obtain values of each two attributes in the different dimensions. The obtained values of different dimensions are marked as subscripts of a multi-dimensional array. A check matrix is constructed by using the multi-dimensional array. A value of an element in the check matrix is extracted. A determination conclusion about rational use of the medicine is outputted based on the value of the obtained element, thereby helping a doctor to rapidly check rational use of a medicine, so as to achieve safe, reasonable and effective use of various medicines to patients.

### BRIEF DESCRIPTION OF THE DRAWINGS

In order to more clearly illustrate embodiments of the present disclosure, the drawings to be used in the description of the embodiments are briefly described below. Apparently, the drawings in the following description show only some embodiments of the present disclosure, and other drawings may be obtained by those skilled in the art from the given drawings without any creative work.

FIG. 1 is a flow chart simply illustrating a method based on matrix check for rapidly determining rational use of a medicine according to the present disclosure.

FIG. 2 is a diagram simply illustrating a system based on matrix check for rapidly determining rational use of a medicine according to the present disclosure.

### DETAILED DESCRIPTION OF EMBODIMENTS

The technical solutions in the embodiments of the present disclosure are described clearly and completely in conjunction with the drawings in the embodiments of the disclosure hereinafter. It is apparent that the described embodiments are only some rather than all embodiments of the present disclosure. Based on the embodiments of the present disclosure, all other embodiments obtained by those skilled in the art without any creative efforts fall within the protection scope of the present disclosure.

In order to make the skilled in the art understand the technical solutions of the present disclosure better, the present disclosure is described in further detail below in conjunction with the drawings and specific embodiments.

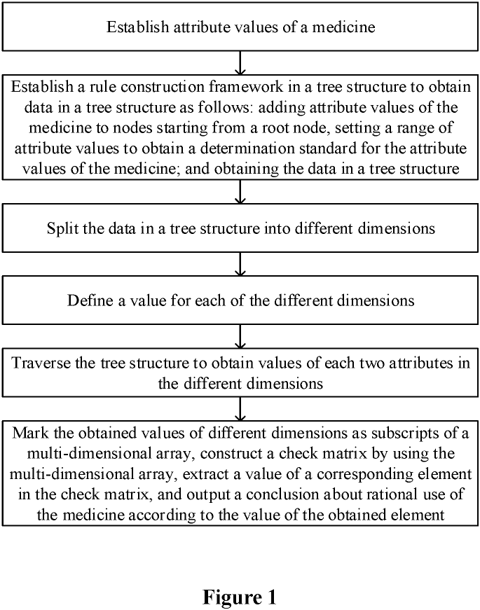

As shown in FIG. 1, a method based on matrix check for rapidly determining rational use of a medicine is provided according to a first embodiment of the present disclosure. The method includes the following steps S11 to S16.

In step S11, attribute values of the medicine are established.

In step S12, a rule construction framework in a tree structure is established to obtain data in a tree structure as follows. The medicine attribute values are added to nodes starting from a root node. A range of attribute values is set, to obtain a determination standard for the attribute values. The data in a tree structure is obtained.

In step S13, the data in a tree structure is split according to different dimensions.

In step S14, a value is defined for each of the different dimensions.

In step S15, the tree structure is traversed to obtain values of each two attributes in the different dimensions.

In step S16, the obtained values of different dimensions are marked as subscripts of a multi-dimensional array. A check matrix is constructed by using the multi-dimensional array. A value of an element in the check matrix is extracted. A determination conclusion about rational use of the medicine is outputted based on the value of the obtained element.

In step S11, the medicine attribute values include rules of use of the medicine and conditions of use of the medicine. The construction of rules of use of a medicine is extremely complex due to strong professionalism in the medical field. Therefore, a reference file access may be added in a rule interface to assist in construction of rules of use of a medicine. In addition, due to the large number of medicine rules, convenient operations including copying and pasting, zooming in and out of nodes, creating a public node, and querying rules of a current medicine in other medicines can be realized in user operations.

The determination standard for the attribute values of the medicine includes: a text class, a numerical interval class and an object class. Attributes of a medicine are diverse and complex, resulting in a large number of table structures defined for storing data.

In an embodiment, the data is stored in a semi-structured and semi-serialized manner. The same structure is defined as a table field, and the attribute values are serialized into a unified field, thereby reducing redundant storage, reducing structure complexity of a database, reducing associated query, and improving the query efficiency.

The data in a tree structure is split according to the following dimensions 1 to 4.

The dimension 1 represents a node relationship, including: a peer node relationship, a sibling node relationship, a parent-child node relationship, and a condition-rule node relationship.

The dimension 2 represents a type of each attribute values, including: a character type, a numerical type, an object type, and a combination type. The character type is for representing an attribute value with a string set. The numerical type is for representing an attribute value with an interval. The object type is for representing an attribute value with multiple groups of key-value. The combination type is for representing an attribute value with multiple groups of intervals or sets.

The dimension 3 represents an operator relationship, including: element of & not element of, element of & element of, not element of & not element of, and element of & equality.

The dimension 4 represents an interval or set relationship, including: non-intersection, intersection, subset, equality and the like.

The value is defined for each of the different dimensions as follows.

For the dimension 1, a value is directly assigned to each relationship. For example, the relationship is represented by a number, such as a peer node relationship=1, a sibling node relationship=2, a parent-child node relationship=3, a condition-rule node relationship=4, no relationship=5, to obtain a relationship dimension R1: [1,2,3,4,5].

For the dimension 2, coding is performed for different types of attribute values, such as: symbolic attribute values, for example, diagnosis is an element of {“cold”, “upper respiratory tract infection”}; numerical attribute values, for example, age is an element of (0,18]; object attribute values, for example, medicine attribute is an element of {“antibacterial medicine”: true, “type”: “western medicine”, “basic medicine”: true...}; and combined attribute values, for example, kidney damage is an element of {“degree”: [“severe”, “incomplete”], “Scr”:(10, 20], “Ccr”:(5, 10], “eGFR”:(3, 6]}. The various types are coded to obtain an array R2:[1,2,3,4].

For the dimension 3, values are assigned to operators in censorship rules. Numerical tokens of two operators are added by concatenating strings. The combined numbers are sorted in ascending order to obtain an ordered array. A subscript of the ordered array represents a dimension value of operation relation, to obtain an array R3.

It can be seen from the dimension 2 that each attribute has an assignment operator, and the operators used in the censorship rules includes ∈, ∉, ⊇, =, ≠ represented by numbers 1, 2, 3, 4 and 5 respectively. Logics of the operator relationship between two attribute rules are compared, and numerical tokens of two operators are added by concatenating strings, for example, ∈&∉ = “1″+“2” = 12, and ∈&∈ = “1″+“1” = 11, so that the finally formed number is unique. Then, all the combined numbers are sorted in ascending order to obtain an ordered array arr[11, 12, 13, 14, 15, 21, 22,..., 55]. A subscript of the ordered array represents a dimension value of operation relation, that is, arr[0]=11, arr[1]=12,..., and a continuous dimension R3 is finally obtained.

For the dimension 4, a value is assigned to a relationship between two values of a same attribute to obtain R4 [1, 2, 3, 4]. For example, age 1∈(0, 3], age 2∈(2, 5], the two intervals has an intersection of (2, 3], and the relationship is marked as intersection. The intersection of attributes of an object type is defined as: in a case that a group of key-values are the same in two value objects of an attribute of the same object type, the relationship is marked as intersection. For example, a combination medicine attribute 1∈{“antibacterial medicine”: true, “type”: “western medicine”, “basic medicine”: true,... }, and a combined medicine attribute 2∈ { “antibacterial medicine”: true, “type”: “western medicine”, “basic medicine”: false,...}. The combination medicine attribute 1 and 2 have the same attribute “western medicine” and “antibacterial medicine”, and thus the relationship therebetween is marked as intersection.

The step S16 includes: calculating values in the 4 dimensions; acquiring element values of the 4-dimensional array; inferring a verification result based on the element values of the array, and outputting the determination conclusion.

For example, if it is determined in the dimension 1 that two attribute rules have the peer node relationship, R1=1 is obtained. If it is determined in the dimension 2 that the attribute value is in the character type, R2 is obtained. If it is determined in the dimension 3 that the logic of the operator relationship between the two attribute rules is element of & element of, a first element in the ordered array is arr[0]=11, and thus R3=1 is obtained. If it is determined in dimension 4 that two attribute rules have intersection, R4 is obtained. Values of the four dimensions are calculated by encoding, to obtain a first array element: arr[0][0][0][1]=2. That is, the obtained information includes: peer node, the same type, element of & not element of, and intersection, and then an inferred result of “repetition” is obtained and a determination conclusion is outputted. If an obtained array element is arr[0][0][1][1]=3, that is, the obtained information includes: parent-child node, the same type, element of & element of, and intersection, then an inferred result of “conflict” is obtained and a determination conclusion is outputted..

In the present application, the problems of low efficiency and low degree of freedom in the existing determination of prescription are solved by a method based on matrix check. With the method, attribute values of a medicine including rules of use of the medicine and conditions of use of the medicine are established, and a rule construction framework in a tree structure is established to obtain data in a tree structure as follows. The medicine attribute values are added to nodes starting from a root node. A range of attribute values is set, to obtain a determination standard for the attribute values. The data in a tree structure is obtained. the data in a tree structure is split according to different dimensions. a value is defined for each of the different dimensions. The tree structure is traversed to obtain values of each two attributes in the different dimensions. The obtained values of different dimensions are marked as subscripts of a multi-dimensional array. A check matrix is constructed by using the multi-dimensional array. A value of an element in the check matrix is extracted. A determination conclusion about rational use of the medicine is outputted based on the value of the obtained element, thereby helping a doctor to rapidly check rational use of a medicine in a prescription, so as to achieve safe, reasonable and effective use of various medicines to patients.

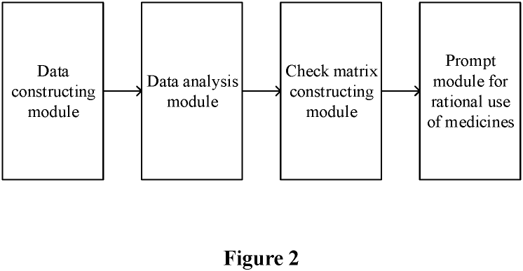

As shown in FIG. 2, a system based on matrix check for rapidly determining rational use of a medicine is provided according to a second embodiment of the present disclosure. The system includes a data establishing module, a data analysis module, a check matrix constructing module and a prompt module for rational use of medicines.

The data constructing module is configured to establish a rule construction framework in a tree structure to obtain data in a tree structure.

The data analysis module is configured to split the data in the tree structure according to different dimensions, define a value for each of the different dimensions, and traverse the tree structure to obtain values of each two attributes in the different dimensions.

The check matrix constructing module is configured to mark the obtained values of different dimensions as subscripts of a multi-dimensional array, and construct a check matrix by using the multi-dimensional array.

The prompt module for rational use of medicines is configured to extract a value of an element in the check matrix, and output a determination conclusion about rational use of the medicine based on the value of the obtained element.

The system based on matrix check for rapidly determining rational use of a medicine according to the embodiment of the present disclosure is described in detail above. The embodiments in this specification are described in a progressive manner, each of the embodiments emphasizes the differences from others, and the same or similar parts among the embodiments may be referred to each other. Since the device disclosed in the embodiments corresponds to the method disclosed therein, the description of the device is relatively simple, and for relevant matters references may be made to the description of the method. It should be noted that several improvements and modifications may be made to the present disclosure by those skilled in the art, without departing from the principle of the present disclosure, and these improvements and modifications also fall within the protection scope of the claims of the present disclosure.

1. A method based on matrix check for rapidly determining rational use of a medicine, comprising:
establishing attribute values of the medicine;
establishing a rule construction framework in a tree structure to obtain data in a tree structure, wherein the establishing a rule construction framework to obtain data in a tree structure comprises: adding the attribute values of the medicine to nodes starting from a root node, setting a range of the attribute values to obtain a determination standard for the attribute values of the medicine, and obtaining the data in a tree structure;
splitting the data in the tree structure according to different dimensions;
defining a value for each of the different dimensions;
traversing the tree structure to obtain values of each two attributes in the different dimensions; and
marking the obtained values in the different dimensions as subscripts of a multi-dimensional array, constructing a check matrix by using the multi-dimensional array, extracting a value of an element in the check matrix, and outputting a determination conclusion about rational use of the medicine based on the value of the obtained element.

2. The method based on matrix check for rapidly determining rational use of a medicine according to claim 1, wherein the attribute values of the medicine comprise rules of use of the medicine and conditions of use of the medicine.

3. The method based on matrix check for rapidly determining rational use of a medicine according to claim 2, wherein the determination standard for the attribute values of the medicine comprises: a text class, a numerical interval class and an object class.

4. The method based on matrix check for rapidly determining rational use of a medicine according to claim 2, wherein the different dimensions comprise:
a dimension 1 representing a node relationship, comprising: a peer node relationship, a sibling node relationship, a parent-child node relationship, and a condition-rule node relationship;
a dimension 2 representing a type of each of the attribute values, comprising: a character type, a numerical type, an object type, and a combination type;
a dimension 3 representing an operator relationship, comprising: element of & not element of, element of & element of, not element of & not element of, and element of & equality; and
a dimension 4 representing an interval or set relationship, comprising: non-intersection, intersection, subset, and equality.

5. The method based on matrix check for rapidly determining rational use of a medicine according to claim 2, wherein the defining a value for each of the different dimensions comprises:
for a dimension 1, directly assigning a value to each relationship, to obtain an array R1;
for a dimension 2, performing coding for different types of the attribute values to obtain an array R2;
for a dimension 3, assigning values to operators in censorship rules, adding numerical tokens of two operators by concatenating strings, sorting combined numbers in ascending order to obtain an ordered array, representing a dimension value of an operation relationship with a subscript of the ordered array, to obtain an array R3; and
for a dimension 4, assigning a value to a relationship between two values of a same attribute to obtain an array R4.

6. The method based on matrix check for rapidly determining rational use of a medicine according to claim 2, wherein the marking the obtained values in the different dimensions as subscripts of a multi-dimensional array, constructing a check matrix by using the multi-dimensional array, extracting a value of an element in the check matrix, and outputting a determination conclusion about rational use of the medicine based on the value of the obtained element comprises:
calculating values in 4 dimensions;
acquiring element values of a 4-dimensional array; and
inferring a verification result based on the element values of the array, and outputting the determination conclusion.

7. A system based on matrix check for rapidly determining rational use of a medicine, comprising:
a data constructing module configured to establish a rule construction framework in a tree structure to obtain data in a tree structure;
a data analysis module configured to split the data in the tree structure according to different dimensions, define a value for each of the different dimensions, and traverse the tree structure to obtain values of each two attributes in the different dimensions;
a check matrix constructing module configured to mark obtained values in the different dimensions as subscripts of a multi-dimensional array, and construct a check matrix by using the multi-dimensional array; and
a prompt module for rational use of medicines configured to extract a value of an element in the check matrix, and output a determination conclusion about rational use of the medicine based on the value of the obtained element.
